(19) United States
(12) Patent Application Publication
(10) Pub. No.: US 2025/0025676 A1
(43) Pub. Date: Jan. 23, 2025

(54) FLOW RESTRICTION ADAPTER FOR RESTRICTING HEMOLYSIS

(71) Applicant: CareFusion 303, Inc., San Diego, CA (US)

(72) Inventors: Ryan CALLAHAN, Long Beach, CA (US); Seyed Alireza SALEHI BOROJERDI, Aliso Viejo, CA (US)

(21) Appl. No.: 18/353,606

(22) Filed: Jul. 17, 2023

(51) Int. Cl. A61M 39/10

(52) U.S. Cl. CPC A61M 39/10; A61M 2039/1077; A61M 2205/3334

(57) ABSTRACT

Flow restricting adapters may include a first and second connector portions, to permit coupling of the adapter restricting flow between a catheter and blood collection device, and a post forming an inner lumen for directing blood through the flow restricting device, the post having a length configured to extend through the first connector portion and be positioned within a portion of the blood collection device, such as the needle of a blood collection tube holder, when the flow restricting device is coupled with the blood collection device, the post having an inner lumen to increase flow resistance and reduce shear stress of blood moving through the flow restricting device, thereby decreasing the incidence of hemolysis.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (PIVC). As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from the skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection device may be used. The blood collection container may include a syringe. Alternatively, the blood collection container may include a test tube with a rubber stopper at one end. In some instances, the test tube has had all or a portion of air removed from the test tube so pressure within the test tube is lower than ambient pressure. Such a blood collection container is often referred to as an internal vacuum or a vacuum tube. A commonly used blood collection container is a Vacutainer® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills until the pressure in the blood collection container equalizes with the pressure in the vein and the flow of blood stops.

In some instances, as blood is drawn into a blood collection container, red blood cells may experience high shear stress and be susceptible to hemolysis due to the flow rate through and geometry of the blood collection system. Hemolysis may result in the rejection and discarding of a blood sample. The blood draw can also result in an area of local negative pressure at the catheter tip, which may lead to catheter tip collapse, vein collapse, or other complications that prevent or restrict blood from filling the blood collection container. Furthermore, blood spillage during and/or after blood draw may occur.

The description provided in the background section should not be assumed to be prior art merely because it is mentioned in or associated with the background section. The background section may include information that describes one or more aspects of the subject technology.

### SUMMARY

Provided herein are embodiments of flow restriction adapters configured to restrict, reduce, or prevent the incidence of hemolysis. In some embodiments, the flow restriction adapters comprise a first connector portion forming a female luer having a first cavity, a second connector portion forming a male luer having a second cavity, a post comprising a proximal end positioned between the male luer and the female luer, a middle portion extending through the first cavity, a distal end positioned distally to the female luer, and an inner lumen extending from the second cavity through the distal end of the post.

Some embodiments provide a flow restriction adapter comprising a first connector portion comprising a first connector end, an inner surface, and a first cavity floor, wherein the inner surface and the first cavity floor form a first cavity a second connector portion comprising a second connector end and an inner surface forming a second cavity, and a post comprising a proximal end at the first cavity floor, a middle portion extending through the first cavity, a distal end positioned distally to the first connector end, and an inner lumen extending from the second cavity through the distal end of the post.

In some embodiments, peripheral intravenous catheter assemblies are provided that are configured to limit hemolysis during drawing of blood from a patient. Some embodiments of these assemblies comprise a blood collection tube and a flow restriction adapter, where a first connector portion of the flow restriction adapter is configured to couple with the blood collection tube such that a tube of the flow restriction adapter extends into the blood collection tube, and a second connector of the flow restriction adapter is couplable with the catheter assembly, and where the tube of the flow restriction adapter provides a narrow fluid flow path for blood moving through the flow restriction adapter, thereby decreasing the incidence of hemolysis by increasing flow resistance and reducing shear stress to the blood moving through the flow restricting device.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a vascular access device including a peripheral intravenous catheter (PIVC) assembly that includes a flow restriction adapter for restricting hemolysis, in accordance with some embodiments of the present disclosure.

FIG. 2 illustrates an elevation view of the flow restriction adapter, in accordance with some embodiments of the present disclosure.

FIG. 3 illustrates a cross-sectional view of the flow restriction adapter of FIG. 2, in accordance with some embodiments of the present disclosure.

FIG. 4A illustrates an elevation view of the flow restriction adapter of FIG. 2 in a first position relative to a fluid collection device, in accordance with some embodiments of the present disclosure.

FIG. 4B illustrates an elevation view of the flow restriction adapter of FIG. 2 in a second position relative to a fluid collection device, in accordance with some embodiments of the present disclosure.

FIG. 5 illustrates a cross-sectional detail view of the flow restriction adapter and the fluid collection device of FIG. 4B.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Blood draw via a vascular access device has drawn increasing attention attributed to minimized needle sticks and improved operation efficiency as compared with traditional blood draw methods with venipuncture. Current blood draw using a peripheral intravenous catheter (PIVC) has seen some challenges, one of the most critical being hemolysis related blood quality. In particular, with currently existing PIVC products in the market, along with the standard connection (such as a short extension set and a needleless connector) and blood collection devices, the shear stress exerted onto red blood cells can cause hemolysis.

Various embodiments of the present disclosure are directed to providing systems and methods to address hemolysis in PIVC blood draw with a hemolysis reduction accessory in the form of a flow restriction adapter, which can be attached to the PIVC or other fluid transfer apparatus to reduce or restrict fluid (e.g., blood) flow therethrough and reduce the risk of hemolysis. The hemolysis-reduction accessory is advantageously compatible with PIVC placement and does not necessitate change to any of the existing operations. The hemolysis-reduction accessory of the various embodiments described herein is potentially applicable to a wide variety of PIVC products and is compatible with existing blood collection devices and infusion components.

Various embodiments of the present disclosure focus on effective flow restriction with the add-on hemolysis-reducing flow restriction adapter that regulates the overall flow rate of the entire fluid path as blood travels through the flow restriction adapter. The flow restriction adapter can be provided alone or either assembled with or co-packaged with any of a PIVC and/or a blood collection device. The clinician may connect a blood collection device to the port or connection portion of the flow restriction adapter and can then draw blood to the intended volume. After blood draw, the clinician may disconnect and discard the flow restriction adapter or leave it coupled with the PIVC. As such, this flow restriction adapter can be used for either single blood draw or stay inline throughout indwell.

The flow restriction adapter and associated blood collection systems of the various embodiments described herein additionally provide further advantages over currently existing blood collection systems. For example, the flow restriction adapter described herein can reduce hemolysis while allowing blood to be drawn through an already-placed PIVC and can be used with existing blood collection containers. Further, the flow restriction adapter described herein is compatible with PIVC connections and allow for seamless blood draw at insertion. Additionally, since the flow restriction adapter can be easily incorporated without any changes to existing PIVC or blood collection containers, there is minimal impact to clinical setting and operations. Additionally, the ability to couple the flow restriction adapter of the present disclosure to existing PIVC systems and blood collection devices or containers provides an efficient and low cost solution for reducing hemolysis.

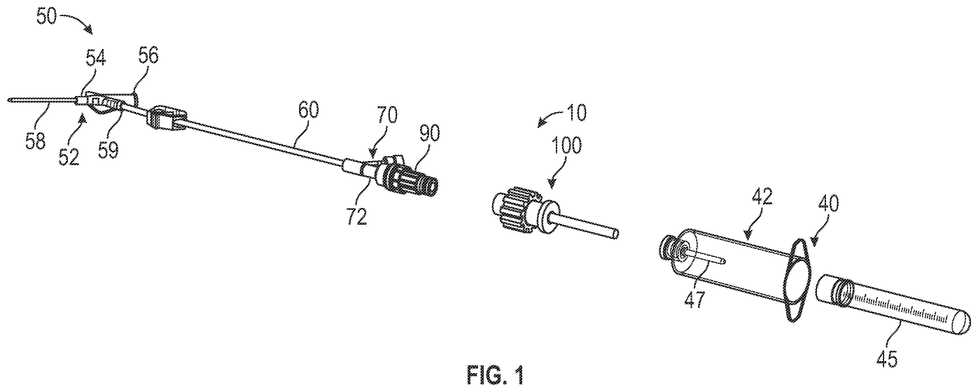

FIG. 1 illustrates a vascular access device 10 including a PIVC assembly 50, as well as a blood collection device 40, and a flow restriction adapter 100, in accordance with some embodiments of the present disclosure. The flow restriction adapter 100 may be configured to facilitate connections between the vascular access device 10 and the blood collection device 40 to reduce a likelihood of hemolysis during movement of blood from the vascular access device 10 to the blood collection device 40.

In some embodiments, the vascular access device 10 may include a catheter assembly 50. The catheter assembly 50 includes a catheter hub 52, which may include a distal end 54, a proximal end 56, and a lumen extending through the distal end and the proximal end. The catheter assembly 50 may further include a catheter 58, which may be secured within the catheter hub 52 and may extend distally from the distal end 54 of the catheter hub 52. In some embodiments, the catheter assembly 50 may be a PIVC.

The blood collection device 40 can include a blood collection tube holder 42 and a blood collection tube 45. The blood collection tube holder 42 includes an inlet port configured to fluidly couple with the PIVC, and a needle 47 configured to be inserted into a blood collection tube 45 that is inserted into the blood collection tube holder 42.

In some embodiments, the catheter assembly 50 may include or correspond to any suitable catheter assembly 50. In some embodiments, the catheter assembly 50 may be integrated and include an extension tube 60, which may extend from and be integrated with a side port 59 of the catheter hub 52. A non-limiting example of an integrated catheter assembly is the BD Nexiva™ Closed IV Catheter system, available from Becton Dickinson and Company. In some embodiments, a proximal end of the extension tube 60 may be coupled to an adapter, such as, for example, a Y-adapter 70. In some embodiments, the flow restriction adapter 100 may be fluidly coupled to the Y-adapter 70.

In some embodiments, the catheter assembly 50 may be non-integrated and may not include the extension tube 60. In these and other embodiments, the flow restriction adapter 100 may be configured to couple to the proximal end 56 of the catheter hub 52 or another suitable portion of the catheter assembly 50. In some embodiments, the flow restriction adapter 100 may be coupled directly to the catheter assembly 50, eliminating the extension tube 60 and providing a compact catheter system.

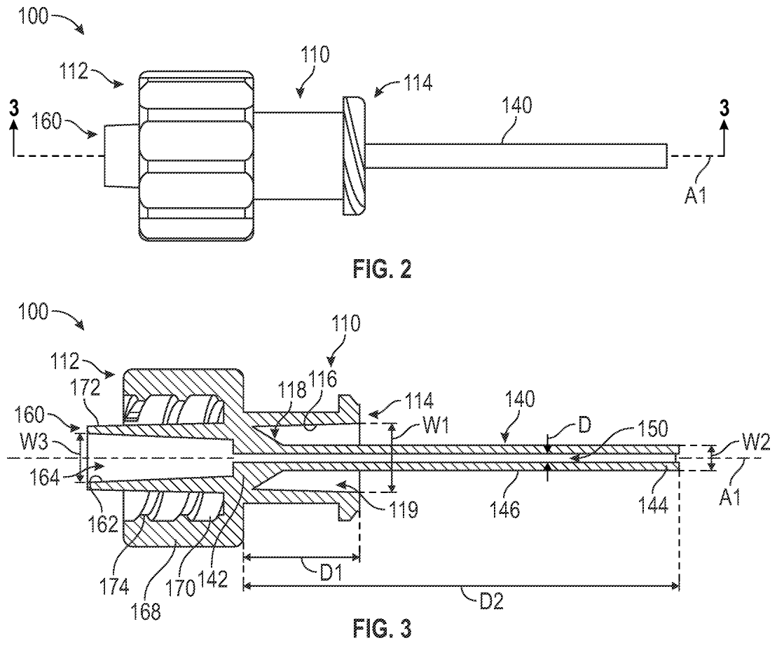

FIG. 2 illustrates an elevation view of the flow restriction adapter 100, and FIG. 3 illustrates a cross-sectional view of the flow restriction adapter 100 of FIG. 2 along the line 3-3. The flow restriction adapter 100 is configured to be fluidly coupled to a blood collection device 40 to restrict a rate of fluid flow through to restrict homolysis. The flow restriction adapter 100 can be coupled directly to a blood collection device 40, such as by coupling to a blood collection tube holder 42, or the flow restriction adapter 100 can be coupled along another portion of a fluid pathway used for blood collection.

To couple the flow restriction adapter 100 to a blood collection device 40 or another portion of a fluid pathway, the flow restriction adapter 100 includes a first connector portion 110 and a second connector portion 112. The first connector portion 110 can couple to a blood collection tube holder 42, and the second connector portion 112 can couple to a Y-adapter 70 or another portion of a catheter assembly 50, as shown, for example, in FIG. 1.

The first and second connector portions 110, 112 are positioned on opposite sides of the flow restriction adapter 100 and are axially aligned along a longitudinal axis A1 defined between the first and second connector portions 110, 112. However, in some embodiments of the present disclosure, it is contemplated that the first and second connector portions 110, 112 can be positioned offset, relative to each other and the longitudinal axis A1. In some embodiments of the present disclosure, the first connector end 114 can be oriented transverse relative to the second connector portion 112 such that a fluid pathway of the first connector portion 110 is transverse, or in a different direction, to the a fluid pathway of the second connector portion 112.

The first connector portion 110 includes a first connector end 114, an inner surface 116, and a first cavity floor 118, which form a first cavity 119 that extends through the first connector end 114. The first cavity 119 of the flow restriction adapter 100 is configured to receive a mating connector therein. The inner surface 116 of the first connector defines a first connector cavity width W1. The first connector cavity width W1 can be configured such that the inner surface 116 of the first connector portion engages against a mating connector that is inserted through the first connector end 114 into the first cavity 119 and forms a fluid tight seal therebetween.

To receive a mating connector into the first cavity of the first connector portion 110, and to form a fluid-tight seal between the first connector portion 110 and the mating connector, the first connector cavity width W1 can, in some embodiments, decrease in a direction from the first connector end 114 toward the first cavity floor 118. In some embodiments of the present disclosure, the first connector portion 110 forms a female luer fitting.

A post 140 extends through the first cavity of the first connector portion 110 and through the first connector end 114. The post 140 is configured to be positioned inside of a needle 47 of a blood collection tube holder 42 when the first connector portion 110 is coupled with a blood collection device 40, such as the BD Vacutainer® LuerLok™ Access Device (LLAD). The post 140 also forms a portion of a fluid passageway through the flow restriction adapter 100 that is configured to reduce the potential occurrence of hemolysis by reducing a flow rate of blood moving through the post 140.

The post 140 includes a proximal end 142, a distal end 144, and a middle portion 146 that extends between the proximal and distal ends 142, 144. The proximal end 142 is located at the first cavity floor 118, and the middle portion 146 of the post extends through the first cavity formed by the first connector portion 110. The middle portion 146 extends entirely through the first connector end 114 so that the distal end 144 of the post is positioned outside of the first connector portion 110.

To provide the post 140 having a distal end 144 that is located outside of the first connector portion 110, i.e., distal to the first connector end 114, the post 140 has a length that is greater than a length of the first connector portion 110. Referring to FIG. 3, the first connector end 114 is positioned a first distance D1 from the first cavity floor 118, and the distal end 144 of the post is a second distance D2 from the first cavity floor 118, where the first distance D1 is less than the second distance D2. In some embodiments of the present disclosure, the first distance D1 is between approximately 10% and approximately 90% of the second distance D2. In some embodiments, the first distance D1 is between approximately 25% and approximately 75% of the second distance D2. In some embodiments, the first distance D1 is approximately 25% of the second distance D2, and in some embodiments, the first distance D1 is approximately 30% of the second distance D2. In instances of the present disclosure, the first distance D1 is approximately 50% or less than the second distance D2.

The outer surface of the post 140 defines a cross-sectional profile that is transverse relative to the longitudinal axis A1, and the cross-sectional profile of the post defines a post width W2. The post width W2 is less than the first connector cavity width W1 such that the outer surface of the post 140 is spaced apart from the inner surface 116 of the first connector portion 110. Thus, when the first connector portion 110 of the flow restriction adapter is coupled with a mating connector, such as blood collection device 40, a portion of the mating connector is received between the outer surface of the post 140 and the inner surface 116 of the first connector portion 110. In some embodiments of the present disclosure, the post width W2 decreases in a direction from the proximal end 142 toward the distal end 144 of the post.

To form a fluid pathway through the flow restriction adapter 100, the post 140 includes an inner lumen 150 that extends through the post 140 to the second connector portion 112. The inner lumen 150 can extend through the distal end 144 of the post, or through another portion of the post 140, such as the middle portion 146 of the post. The inner lumen 150 defines the fluid pathway, or a portion thereof, through the flow restriction adapter 100. The fluid pathway along the inner lumen 150 has an inner lumen width D and an inner lumen length, which can be equal to or approximately equal to the second distance D2.

The inner lumen 150 is fluidly coupled with the second connector portion 112 so that a fluid (i.e., blood) can move from the second connector portion 112 and through the post 140. The second connector portion 112 includes a second connector end 160 and an inner surface 162 forming a second cavity 164. The second connector portion 112 is configured to couple with a mating connector, such as a PIVC, and direct fluid (e.g., blood) into the inner lumen 150 of the post.

The inner surface 162 of the second cavity 164 defines a second cavity width W3. The second cavity width W3 is greater than the inner lumen width D; however, the present disclosure also contemplated embodiments in which the second cavity width W3 is equal to the inner lumen width D. In some embodiments of the present disclosure, the second cavity width W3 decreases in a direction from the second connector end 160 toward the first connector portion 110.

The second connector portion 112 includes a collar 168 that extends circumferentially around the second connector portion 112 and along a length of the second connector portion 112 toward the second connector end 160. The collar 168 has an inner surface 170 that is spaced apart from outer surface 172 of the second connector portion to receive at least a portion of a mating connector. The collar 168 can also include a thread 174 that extends radially inward to engage with a thread of the mating connector. In some embodiments of the present disclosure, the second connector portion 112 forms a male luer fitting.

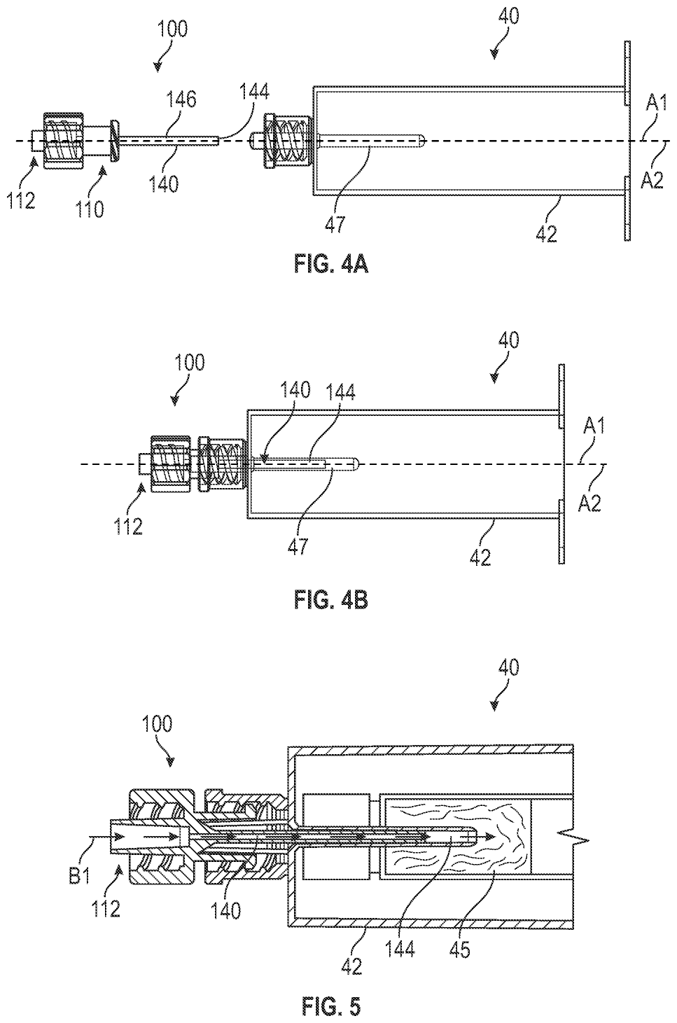

Coupling the flow restriction adapter 100 to a blood collection device 40 is illustrated in FIGS. 4A and 4B. In FIG. 4A, the flow restriction adapter 100 is in a first position in which the flow restriction adapter 100 is spaced apart from the blood collection device 40. The flow restriction adapter 100 is oriented with the longitudinal axis A1 of the flow restriction adapter 100 and is aligned or coaxial with a longitudinal axis A2 of the needle 47. The flow restriction adapter 100 and/or the blood collection device 40 can be moved toward each other so that the post 140 is inserted into the lumen of the needle 47 of the blood collection tube holder 42.

As the flow restriction adapter 100 and the blood collection device 40 move toward each other, the post 140 extends within the needle 47 and the first connector portion 110 can couple with the mating connector of the blood collection tube holder 42. In the embodiment shown in FIGS. 4A-5, the first connector portion 110 is a female luer that is coupled to a male luer of the blood collection tube holder 42 by threads of the first connector portion 110 and the blood collection tube holder 42.

Referring to FIG. 5, a cross-sectional detail view of the flow restriction adapter 100 and the blood collection device of FIG. 4B are illustrated with a blood collection tube 45 positioned within the blood collection tube holder 42. The movement of blood is illustrated by arrows B1, which moves through the inner lumen 150 in a direction from the second connector portion 112 toward the distal end 144 of the post. The inner lumen 150 of the post 140 provides a fluid pathway with a narrow diameter or cross-section to induce hydraulic resistance and reduce or restrict homolysis of the blood moving therethrough. Further, the features of the flow restriction adapter 100 provide a device for restricting fluid flow to restrict homolysis that can be implemented with existing devices. Additionally, the flow restriction adapter 100 provides features for restricting flow for restricting hemolysis while reducing the risk of infection since the post 140 is not required to be inserted into the PIVC.

The flow restriction adapters of the present disclosure additionally provide further advantages over currently existing blood collection systems. For example, the flow restriction adapters described herein allow for hemolysis-reduction function to be integrated for PIVC blood draw. Further, the flow restriction adapters described herein are compatible with PIVC placement and allow for seamless blood draw at insertion. In some embodiments, the flow restriction adapters have the potential to stay inline throughout PIVC indwell for multiple blood draws. Additionally, since the flow restriction adapters can be an add-on which can be easily incorporated without any changes to an existing PIVC, there is minimal impact to clinical setting and operations.

An optimized fluid pathway, which can include the inner lumen 150, can be configured for providing a restricted flow rate for reducing hemolysis, and can have features including, but not limited to, a tubular fluid pathway, a cannula, a lumen, a continuous non-linear channel, a continuous linear channel, a groove, a fluid channel, and the like.

The fluid pathway can have a length that is selected based on one or more of the following: a gauge of a particular catheter, a particular catheter assembly configuration, or a clinical setup. In some embodiments, the optimized fluid pathway may include a length L from the first connector portion 110 to the second connector portion 112. In some embodiments, the optimized fluid pathway may include an inner diameter D. In some instances of the present disclosure, the optimized fluid pathway has a length L that is from the first connector end 114 to the first cavity floor 118.

Fluid flow in the fluid pathway therethrough can be analyzed using Poiseuille's equation:

$$Q = \frac{π {D}^{4} Δ P}{128 μ L} = \frac{Δ P}{{R}_{f}}$$

where ΔP is a change in pressure gradient across the length of the fluid pathway, D and L are the inner diameter and length, respectively, of the fluid pathway, μ is the viscosity of a fluid, and

$${R}_{f} = \frac{128 μ L}{π {D}^{4}}$$

is the fluid resistance. Since μ is the viscosity of the fluid and not part of the extension tube geometry, a geometric factor G_{f} is defined such that R_{f} (the fluid resistance) is

$${R}_{f} = \frac{128 μ}{π} {G}_{f} ,$$

$$where$$

$${G}_{f} = \frac{L}{{D}^{4}} .$$

In some embodiments, the fluid pathway may have multiple sections with lengths (L1, L2, L3) and inner diameters (D1, D2, D3). The geometric factor is then:

$${G}_{f} = {\int }_{0}^{L} \frac{dl}{D {( l )}^{4}}$$

In some embodiments, the optimized fluid pathway may have a cross section that is not circular or a complicated inner profile. The geometric factor can be determined by measuring the flow rate (Q) at given pressure (ΔP) with known viscosity (μ) fluid:

$${G}_{f} = \frac{πΔ P}{128 μ Q}$$

The G_{f} value of the optimized fluid pathway may be selected to reduce the max shear stress for each catheter gauge to be the same or less than the max shear stress of a BD 21G Vacutainer® UltraTouch™ push button blood collection set. In some embodiments, G_{f} value of the fluid pathway may be selected to reduce the max shear stress for each catheter gauge to be the same or less than the max shear stress of a BD 25G Vacutainer® UltraTouch™ push button blood collection set.

In some embodiments, and by way of non-limiting example, the fluid pathway can have a diameter of approximately 0.014 inches. In another non-limiting example, a cross-sectional area of the fluid pathway is approximately 0.000152 square-inches.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A flow restriction adapter for restricting hemolysis, the flow restriction adapter comprising: a first connector portion forming a female luer having a first cavity; a second connector portion forming a male luer having a second cavity; a post comprising a proximal end positioned between the male luer and the female luer, a middle portion extending through the first cavity, a distal end positioned distally to the female luer, and an inner lumen extending from the second cavity through the distal end of the post.

Clause 2. The flow restriction adapter of Clause 1, wherein the first connector portion comprises a first cavity floor forming a portion of the cavity, an end of the female luer is positioned a first distance form the first cavity floor, and the distal end of the of the post is positioned a second distance from the first cavity floor, and wherein the first distance is less than the second distance.

Clause 3. The flow restriction adapter of Clause 2, wherein the first distance is approximately 50 percent or less than the second distance.

Clause 4. The flow restriction adapter of any of Clauses 1 to 3, wherein: an outer surface of the post defines a cross-sectional profile that is transverse relative to a longitudinal axis of the post extending between the proximal and distal ends of the post, and the cross-sectional profile of the post defines a post width; and the first cavity defines a first connector cavity width, wherein the post width is less than the first connector cavity width.

Clause 5. The flow restriction adapter of Clause 4, wherein the post width decreases in a direction from the first cavity floor toward the distal end of the post.

Clause 6. The flow restriction adapter of any of Clauses 1 to 5, wherein an outer surface of the post is spaced apart from an inner surface of the female luer.

Clause 7. The flow restriction adapter of any of Clauses 1 to 6, wherein an inner surface of the female luer defines a first connector cavity width, and wherein the first connector cavity width decreases in a direction from an end of the female luer toward the first cavity floor.

Clause 8. The flow restriction adapter of any of Clauses 1 to 7, further comprising a collar extending along the male luer, wherein the collar comprises an inner surface spaced apart from the outer surface of the male luer.

Clause 9. The flow restriction adapter of any of Clauses 1 to 8, wherein the inner lumen defines an inner lumen width, and the second cavity defines a second cavity width, and wherein the inner lumen width is less than the second cavity width.

Clause 10. The flow restriction adapter of any of Clauses 1 to 9, wherein the second cavity defines a second cavity width that decreases in a direction from an end of the male luer toward the female luer.

Clause 11. A flow restriction adapter for restricting hemolysis, the flow restriction adapter comprising: a first connector portion comprising a first connector end, an inner surface, and a first cavity floor, wherein the inner surface and the first cavity floor form a first cavity; a second connector portion comprising a second connector end and an inner surface forming a second cavity; and a post comprising a proximal end at the first cavity floor, a middle portion extending through the first cavity, a distal end positioned distally to the first connector end, and an inner lumen extending from the second cavity through the distal end of the post.

Clause 12. The flow restriction adapter of Clause 11, wherein the first connector end is positioned a first distance from the first cavity floor, and the distal end of the post is a second distance from the first cavity floor, and wherein the first distance is less than the second distance.

Clause 13. The flow restriction adapter of Clause 12, wherein the first distance is approximately 50 percent or less than the second distance.

Clause 14. The flow restriction adapter of any of Clauses 11 to 13, wherein: an outer surface of the post defines a cross-sectional profile that is transverse relative to a longitudinal axis of the post extending between the proximal and distal ends of the post, and the cross-sectional profile of the post defines a post width; and the inner surface of the first connector portion defines a first connector cavity width; and wherein the post width is less than the first connector cavity width.

Clause 15. The flow restriction adapter of Clause 14, wherein the post width decreases in a direction from the proximal end of the post toward the distal end of the post.

Clause 16. The flow restriction adapter of any of Clauses 11 to 15, wherein an outer surface of the post is spaced apart from the inner surface of the first connector portion.

Clause 17. The flow restriction adapter of any of Clauses 11 to 16, wherein the inner surface of the first connector portion defines a first connector cavity width, and wherein the first connector cavity width decreases in a direction from the first connector end toward the first cavity floor.

Clause 18. The flow restriction adapter of any of Clauses 11 to 17, further comprising a collar extending along the second connector portion, wherein the collar comprises an inner surface spaced apart from the outer surface of the second connector portion.

Clause 19. The flow restriction adapter of any of Clauses 11 to 18, wherein the inner lumen defines an inner lumen width, and the second cavity defines a second cavity width, and wherein the inner lumen width is less than the second cavity width.

Clause 20. The flow restriction adapter of any of Clauses 11 to 19, wherein the second cavity defines a second cavity width that decreases in a direction from an end of the second connector portion toward the first connector portion.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed, are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A flow restriction adapter for restricting hemolysis, the flow restriction adapter comprising:
a first connector portion forming a female luer having a first cavity;
a second connector portion forming a male luer having a second cavity;
a post comprising a proximal end positioned between the male luer and the female luer, a middle portion extending through the first cavity, a distal end positioned distally to the female luer, and an inner lumen extending from the second cavity through the distal end of the post.

2. The flow restriction adapter of claim 1, wherein the first connector portion comprises a first cavity floor forming a portion of the cavity, an end of the female luer is positioned a first distance form the first cavity floor, and the distal end of the of the post is positioned a second distance from the first cavity floor, and wherein the first distance is less than the second distance.

3. The flow restriction adapter of claim 2, wherein the first distance is approximately 50 percent or less than the second distance.

4. The flow restriction adapter of claim 1, wherein:
an outer surface of the post defines a cross-sectional profile that is transverse relative to a longitudinal axis of the post extending between the proximal and distal ends of the post, and the cross-sectional profile of the post defines a post width; and
the first cavity defines a first connector cavity width, wherein the post width is less than the first connector cavity width.

5. The flow restriction adapter of claim 4, wherein the post width decreases in a direction toward the distal end of the post.

6. The flow restriction adapter of claim 1, wherein an outer surface of the post is spaced apart from an inner surface of the female luer.

7. The flow restriction adapter of claim 1, wherein an inner surface of the female luer defines a first connector cavity width, and wherein the first connector cavity width decreases in a direction away from an end of the female luer.

8. The flow restriction adapter of claim 1, further comprising a collar extending along the male luer, wherein the collar comprises an inner surface spaced apart from the outer surface of the male luer.

9. The flow restriction adapter of claim 1, wherein the inner lumen defines an inner lumen width, and the second cavity defines a second cavity width, and wherein the inner lumen width is less than the second cavity width.

10. The flow restriction adapter of claim 1, wherein the second cavity defines a second cavity width that decreases in a direction from an end of the male luer toward the female luer.

11. A flow restriction adapter for restricting hemolysis, the flow restriction adapter comprising:
a first connector portion comprising a first connector end, an inner surface, and a first cavity floor, wherein the inner surface and the first cavity floor form a first cavity;
a second connector portion comprising a second connector end and an inner surface forming a second cavity; and
a post comprising a proximal end at the first cavity floor, a middle portion extending through the first cavity, a distal end positioned distally to the first connector end, and an inner lumen extending from the second cavity through the distal end of the post.

12. The flow restriction adapter of claim 11, wherein the first connector end is positioned a first distance from the first cavity floor, and the distal end of the post is a second distance from the first cavity floor, and wherein the first distance is less than the second distance.

13. The flow restriction adapter of claim 12, wherein the first distance is approximately 50 percent or less than the second distance.

14. The flow restriction adapter of claim 11, wherein:
an outer surface of the post defines a cross-sectional profile that is transverse relative to a longitudinal axis of the post extending between the proximal and distal ends of the post, and the cross-sectional profile of the post defines a post width; and
the inner surface of the first connector portion defines a first connector cavity width; and
wherein the post width is less than the first connector cavity width.

15. The flow restriction adapter of claim 14, wherein the post width decreases in a direction from the proximal end of the post toward the distal end of the post.

16. The flow restriction adapter of claim 11, wherein an outer surface of the post is spaced apart from the inner surface of the first connector portion.

17. The flow restriction adapter of claim 11, wherein the inner surface of the first connector portion defines a first connector cavity width, and wherein the first connector cavity width decreases in a direction from the first connector end toward the first cavity floor.

18. The flow restriction adapter of claim 11, further comprising a collar extending along the second connector portion, wherein the collar comprises an inner surface spaced apart from the outer surface of the second connector portion.

19. The flow restriction adapter of claim 11, wherein the inner lumen defines an inner lumen width, and the second cavity defines a second cavity width, and wherein the inner lumen width is less than the second cavity width.

20. The flow restriction adapter of claim 11, wherein the second cavity defines a second cavity width that decreases in a direction from an end of the second connector portion toward the first connector portion.
